(19) United States
(12) Patent Application Publication
(10) Pub. No.: US 2025/0302331 A1
(43) Pub. Date: Oct. 2, 2025

(54) PRESSURE SENSOR ARRAY AND RESPIRATORY MONITORING APPARATUS INCLUDING THE SAME

(71) Applicants: Samsung Display Co., Ltd., Yongin-si (KR); POSTECH Research and Business Development Foundation, Pohang-si (KR)

(72) Inventors: Mankyu Jo, Yongin-si (KR); Jaeyoon Sim, Pohang-si (KR); Unyong Jeong, Pohang-si (KR); Seongwon Kim, Yongin-si (KR); Jaehyun Kim, Pohang-si (KR); Junggun Nam, Yongin-si (KR); Heesung Roh, Pohang-si (KR); Sungchan Jo, Yongin-si (KR)

(21) Appl. No.: 19/089,269

(22) Filed: Mar. 25, 2025

(30) Foreign Application Priority Data
Mar. 26, 2024 (KR) 10-2024-0041134

(51) Int. Cl. A61B 5/08; A41D 13/11; A61B 5/00

(52) U.S. Cl. CPC A61B 5/08; A41D 13/11; A61B 5/6803; A61B 5/7225; A61B 5/7228; A61B 5/7445; A61B 2562/0247; A61B 2562/0285; A61B 2562/046

(57) ABSTRACT

A pressure sensor array includes: a first sheet including first electrodes extending in a first direction and spaced from each other along a second direction crossing the first direction; a second sheet including second electrodes extending in the second direction and spaced from each other along the first direction; and a third sheet between the first sheet and the second sheet, and including: a partition wall defining a plurality of holes that are spaced from each other in the first direction and the second direction; and pressure-sensitive layers located on an inner surface of each of the plurality of holes.

### CROSS-REFERENCE TO RELATED APPLICATION

The present application claims priority to and the benefit of Korean Patent Application No. 10-2024-0041134, filed on Mar. 26, 2024, in the Korean Intellectual Property Office, the entire disclosure of which is incorporated by reference herein.

### BACKGROUND

### 1. Field

Aspects of one or more embodiments of the present disclosure relate to a pressure sensor array, and a respiratory monitoring apparatus including the pressure sensor array.

### 2. Description of the Related Art

Due to fine dust and viruses, such as coronavirus, various respiratory diseases are increasing. A user's breathing pattern may be derived and respiratory diseases may be analyzed by using a respiratory monitoring apparatus, which is an electronic apparatus that senses a user's breathing activity. A respiratory monitoring apparatus may include a sensor configured to measure a pressure and the like that vary with a user's breathing, and a circuit configured to operate the sensor. A respiratory monitoring apparatus may be used while attached to a user's nose and mouth.

The above information disclosed in this Background section is for enhancement of understanding of the background of the present disclosure, and therefore, it may contain information that does not constitute prior art.

### SUMMARY

In a respiratory monitoring apparatus according to a comparative example, a user may feel a foreign body sensation when breathing and the respiratory monitoring apparatus interferes with the user's activities.

Embodiments of the present disclosure may be directed to a stretchable pressure sensor array that is changeable in various shapes, and a respiratory monitoring apparatus including the pressure sensor array. However, the aspects and features of the present disclosure are not limited thereto.

Additional aspects and features will be set forth, in part, in the description that follows, and in part, may be apparent from the description, or may be learned by practicing one or more of the presented embodiments of the present disclosure.

According to one or more embodiments of the present disclosure, a pressure sensor array includes: a first sheet including first electrodes extending in a first direction and spaced from each other along a second direction crossing the first direction; a second sheet including second electrodes extending in the second direction and spaced from each other along the first direction; and a third sheet between the first sheet and the second sheet, and including: a partition wall defining a plurality of holes that are spaced from each other in the first direction and the second direction; and pressure-sensitive layers located on an inner surface of each of the plurality of holes.

In an embodiment, each of the first sheet, the second sheet, and the third sheet may include nanofibers.

In an embodiment, each of the first electrodes and the second electrodes may include nanofibers coated with a metal.

In an embodiment, the partition wall may include an elastomer.

In an embodiment, each of the pressure-sensitive layers may include nanofibers coated with conductive particles.

In an embodiment, the nanofibers may be bonded to the conductive particles through imide bonding.

In an embodiment, each of the pressure-sensitive layers may overlap with a corresponding one of a plurality of crossing regions in which the first electrodes cross the second electrodes.

According to one or more embodiments of the present disclosure, a respiratory monitoring apparatus includes: a pressure sensor array configured to sense pressure changes caused by a user's breathing; a transponder configured to supply a voltage to the pressure sensor array, convert a sensing signal received from the pressure sensor array into a data signal, and transmit the converted data signal; a reader configured to supply a voltage to the transponder through inductive coupling, and receive the data signal from the transponder; and a mask portion configured to fix the pressure sensor array and the transponder to the user's face. The pressure sensor array includes: a first sheet including first electrodes extending in a first direction and spaced from each other along a second direction crossing the first direction; a second sheet including second electrodes extending in the second direction and spaced from each other along the first direction; and a third sheet between the first sheet and the second sheet, and including: a partition wall defining a plurality of holes that are spaced from each other in the first direction and the second direction; and pressure-sensitive layers located on an inner surface of each of the plurality of holes.

In an embodiment, each of the first sheet, the second sheet, and the third sheet may include nanofibers.

In an embodiment, each of the first electrodes and the second electrodes may include nanofibers coated with a metal.

In an embodiment, the partition wall may include an elastomer.

In an embodiment, each of the pressure-sensitive layers may include nanofibers coated with conductive particles.

In an embodiment, the nanofibers may be bonded to the conductive particles through imide bonding.

In an embodiment, each of the pressure-sensitive layers may overlap with a corresponding one of a plurality of crossing regions in which the first electrodes cross the second electrodes.

In an embodiment, the transponder may include: a first antenna; a rectifier configured to convert an alternating current voltage received through the first antenna into a direct current voltage; a pressure sensor reader configured to input a driving signal to the pressure sensor array, receive a sensing signal from the pressure sensor array, and convert the sensing signal into a first signal; a data serializer configured to serialize the first signal, convert the serialized first signal into the data signal, and change a voltage level of the first antenna according to the data signal; and a power manager configured to receive the direct current voltage from the rectifier, and distribute the direct current voltage.

In an embodiment, the reader may include: a second antenna; an amplifier configured to generate the alternating current voltage according to an input clock signal; a matching network configured to transfer the alternating current voltage from the amplifier to the second antenna; a signal processor configured to sense a voltage level of the first antenna; and a data demodulator configured to demodulate the data signal from the voltage level of the first antenna.

In an embodiment, the alternating current voltage may include a sinusoidal wave of about 433 MHz.

In an embodiment, the respiratory monitoring apparatus may further include a display connected to the mask portion, and configured to display a user's respiratory information.

In an embodiment, the display may include a reflective display panel.

In an embodiment, the reader may be configured to generate, based on the data signal, a control signal including the user's respiratory information, and transfer 1 the control signal to the transponder, and the transponder may be configured to transfer the control signal to the display.

However, the present disclosure is not limited to the above aspects and features, and the above and additional aspects and features will be set forth, in part, in the detailed description that follows with reference to the drawings, and in part, may be apparent therefrom, or may be learned by practicing one or more of the presented embodiments of the present disclosure.

### BRIEF DESCRIPTION OF THE DRAWINGS

The above and other aspects and features of the present disclosure will be more clearly understood from the following detailed description of the illustrative, non-limiting embodiments with reference to the accompanying drawings, in which:

FIG. 1 is a schematic plan view of a pressure sensor array according to an embodiment;

FIG. 2 is a schematic plan view of a first sheet layer according to an embodiment;

FIG. 3 is a schematic plan view of a second sheet layer according to an embodiment;

FIG. 4 is a schematic enlarged plan view of the region II of the first sheet layer shown in FIG. 2;

FIG. 5 is a schematic plan view of a third sheet layer according to an embodiment;

FIG. 6A is a view illustrating conductive particles;

FIG. 6B is a view illustrating nanofibers coated with conductive particles;

FIG. 7A is a schematic cross-sectional view of a pressure sensor array according to an embodiment;

FIG. 7B is a view illustrating a case where a pressure is applied to the pressure sensor array of FIG. 7A;

FIG. 8 is a schematic view of a portion of a respiratory monitoring apparatus according to an embodiment;

FIG. 9 is a schematic block view of a respiratory monitoring apparatus according to an embodiment;

FIG. 10 is a view of a transponder and a reader;

FIG. 11A is a graph showing a relationship between an elongation and a rate of change in resistance of nanofibers coated with a metal;

FIG. 11B is a graph showing a relationship between the number of stretching repetitions and a rate of change in resistance of nanofibers coated with a metal;

FIG. 12A is a graph showing a relationship between a pressure and a rate of change in current in each of an unstretched pressure sensor and a stretched pressure sensor;

FIG. 12B is a graph showing a pressure and a rate of change in current of a pressure sensor measured in real time;

FIG. 12C is a graph showing a relationship between a loading cycle and a rate of change in current of an unstretched pressure sensor;

FIG. 12D is a graph showing a relationship between a loading cycle and a rate of change in current of a stretched pressure sensor; and

FIGS. 13A-13F are graphs showing a pressure change in a pressure sensor array measured using a respiratory monitoring apparatus according to one or more embodiments.

### DETAILED DESCRIPTION

Hereinafter, embodiments will be described in more detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, redundant description thereof may not be repeated.

When a certain embodiment may be implemented differently, a specific process order may be different from the described order. For example, two consecutively described processes may be performed at the same or substantially at the same time, or may be performed in an order opposite to the described order.

Further, as would be understood by a person having ordinary skill in the art, in view of the present disclosure in its entirety, each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner, unless otherwise stated or implied.

In the drawings, the relative sizes, thicknesses, and ratios of elements, layers, and regions may be exaggerated and/or simplified for clarity. Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

Further, it should be expected that the shapes shown in the figures may vary in practice depending, for example, on tolerances and/or manufacturing techniques. Accordingly, the embodiments of the present disclosure should not be construed as being limited to the specific shapes shown in the figures, and should be construed considering changes in shapes that may occur, for example, as a result of manufacturing. As such, the shapes shown in the drawings may not depict the actual shapes of areas of the device, and the present disclosure is not limited thereto.

In the figures, the x-axis, the y-axis, and the z-axis are not limited to three axes of the rectangular coordinate system, and may be interpreted in a broader sense. For example, the x-axis, the y-axis, and the z-axis may be perpendicular to or substantially perpendicular to one another, or may represent different directions from each other that are not perpendicular to one another.

As used herein, the phrases “on a plan view” and “in a plan view” may refer to a view of an objective portion from above (e.g., viewed in a direction perpendicular to or substantially perpendicular to the upper surface of a substrate). The phrases, “on a cross-sectional view” and “in a cross-sectional view” may refer to a cross-section of an objective portion taken vertically and is viewed from a lateral side.

As used herein, when a first element is referred to as “overlaps” or “overlapping” with a second element, the first element may be arranged above or below (e.g., under) the second element to at least partially overlap with the second element in a plan view.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “on,” “connected to,” or “coupled to” another element or layer, it can be directly on, connected to, or coupled to the other element or layer, or one or more intervening elements or layers may be present. Similarly, when a layer, an area, or an element is referred to as being “electrically connected” to another layer, area, or element, it may be directly electrically connected to the other layer, area, or element, and/or may be indirectly electrically connected with one or more intervening layers, areas, or elements therebetween. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” “including,” “has,” “have,” and “having,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. For example, the expression “A and/or B” denotes A, B, or A and B. Expressions such as “at least one of,” when preceding a list of elements, modify the entire list of elements and do not modify the individual elements of the list. For example, the expression “at least one of a, b, or c,” “at least one of a, b, and c,” and “at least one selected from the group consisting of a, b, and c” indicates only a, only b, only c, both a and b, both a and c, both b and c, all of a, b, and c, or variations thereof.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

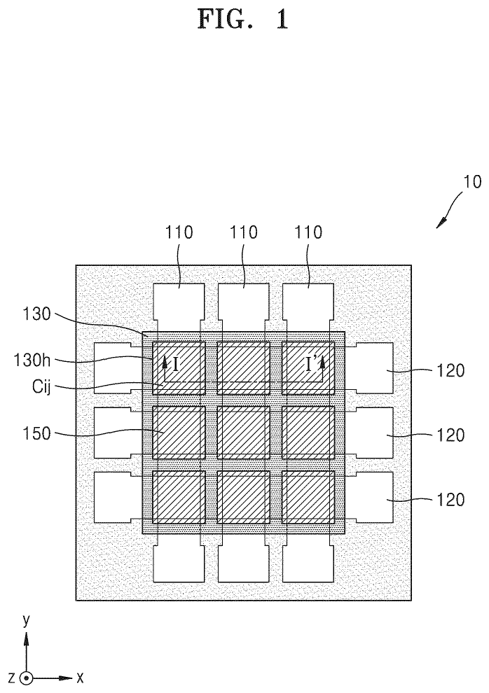

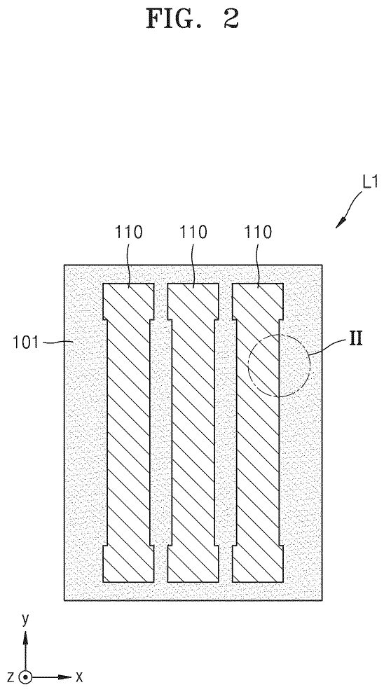

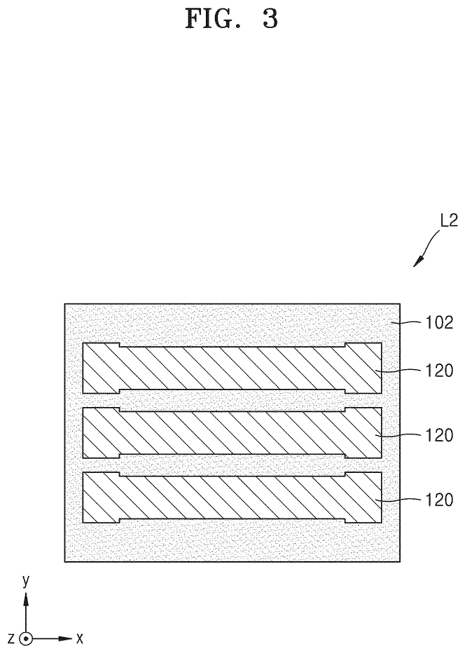

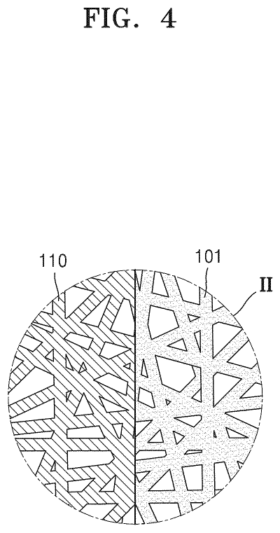

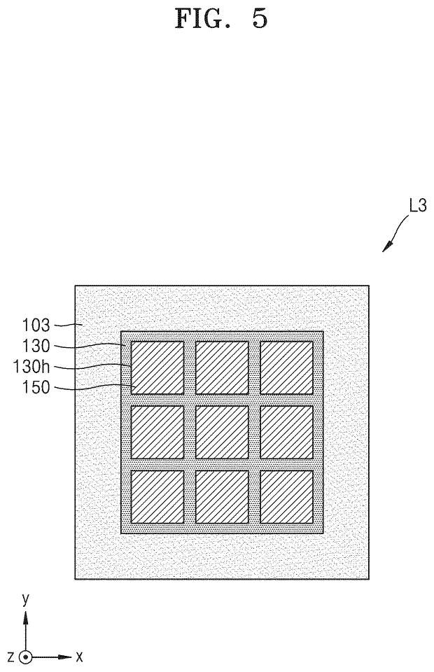

FIG. 1 is a schematic plan view of a pressure sensor array according to an embodiment. FIG. 2 is a schematic plan view of a first sheet layer according to an embodiment. FIG. 3 is a schematic plan view of a second sheet layer according to an embodiment. FIG. 4 is a schematic enlarged plan view of the region II of the first sheet layer shown in FIG. 2. FIG. 5 is a schematic plan view of a third sheet layer according to an embodiment.

Referring to FIGS. 1 to 5, a pressure sensor array 10 according to an embodiment may include first electrodes 110, second electrodes 120 crossing the first electrodes 110, a partition wall 130 defining a plurality of holes 130h, and pressure-sensitive layers 150 disposed in each of the plurality of holes 130h.

The pressure sensor array 10 may have a multi-layered structure in which a plurality of nanofiber sheet layers overlap with each other. As an example, the pressure 1 sensor array 10 may include a first layer L1 including the first electrodes 110, a second layer L2 including the second electrodes 120, and a third layer L3 including the partition wall 130 and the pressure-sensitive layers 150. The first layer L1 may be disposed on the second layer L2, and the third layer L3 may be disposed between the first layer L1 and the second layer L2. FIG. 1 shows that the first layer L1, the second layer L2, and the third layer L3 overlap with each other to illustrate the positional relationship of each element of the pressure sensor array 10.

Referring to FIG. 2, the first layer L1 may include a first nanofiber sheet 101 and the first electrodes 110. Each of the first electrodes 110 may extend in a first direction (e.g., the y direction), and may be spaced apart from each other in a second direction (e.g., the x direction) crossing the first direction (e.g., the y direction).

Referring to FIG. 3, the second layer L2 may include a second nanofiber sheet 102 and the second electrodes 120. Each of the second electrodes 120 may extend in the second direction (e.g., the x direction), and may be spaced apart from each other in the first direction (e.g., the y direction).

Each of the first electrodes 110 and the second electrodes 120 may include nanofibers coated with a metal. In an embodiment, as shown in FIG. 4, the first electrodes 110 may be formed by coating a portion of the first nanofiber sheet 101 with a metal material. Likewise, the second electrodes 120 may be formed by coating a portion of the second nanofiber sheet 102 with a metal material.

The first nanofiber sheet 101 and the second nanofiber sheet 102 may be integrated nanofibers formed using electrospinning, and may have a plurality of holes through which a gas or a fluid may pass. In an embodiment, the nanofibers may include polyurethane, polystyrene-block-poly(ethylene butylene)-block-polystyrene, polystyrene, polyvinyl chloride, or any suitable mixtures thereof. In an embodiment, the nanofibers may be polystyrene-block-poly(ethylene butylene)-block-polystyrene grafted with maleic anhydride (SEBS-g-MA).

Metal materials may be coated on nanofibers by various suitable methods, such as chemical vapor deposition and sputtering. In an embodiment, the metal material may include gold (Au), silver (Ag), platinum (Pt), aluminum (Al), titanium (Ti), molybdenum (Mo), and/or the like.

Because the first electrodes 110 are formed by coating the metal materials on a portion of the first nanofiber sheet 101, and the second electrodes 120 are formed by coating the metal materials on a portion of the second nanofiber sheet 102, a configuration of the electrodes may be maintained even in a case where stretching of the pressure sensor array 10 is repeated or a strong pressure is applied. In addition, because the first electrodes 110 and the second electrodes 120 may have a network structure formed by integrating nanofibers coated with a metal material, a change in resistance may be small (e.g., may be very small) even when the pressure sensor array 10 is stretched. In an embodiment, at least a portion of the first nanofiber sheet 101 on which the first electrodes 110 are not formed, or at least a portion of the second nanofiber sheet 102 on which the second electrodes 120 are not formed, may be removed.

Referring to FIG. 5, the third layer L3 may include the third nanofiber sheet 103, the partition wall 130, and the pressure-sensitive layers 150. The partition wall 130 may form a mesh structure defining the plurality of holes 130h. Although the partition wall 130 defines the holes 130h, each having an approximately quadrangular shape in a plan view in FIGS. 1 to 5, the present disclosure is not limited thereto. In another embodiment, in a plan view, the holes 130h defined by the partition wall 130 may have other suitable polygonal shapes, a circular shape, an elliptical shape, or an irregular shape.

The partition wall 130 may include an insulating elastomer. The insulating elastomer may be a silicone-based elastomer such as polydimethylsiloxane, a styrene-based elastomer, an olefin-based elastomer, polyurethane, or any suitable mixtures thereof. The partition wall 130 may be formed by filling a partial hole of the third nanofiber sheet 103 with an insulating elastomer. The partition wall 130 may have a continuous or substantially continuous surface without holes.

The pressure-sensitive layers 150 may be disposed on the inner surface of each of the plurality of holes 130h. The pressure-sensitive layers 150 may be spaced apart and separated from each other along the first direction (e.g., the y direction) and the second direction (e.g., the x direction) with the partition wall 130 therebetween. The first layer L1, the second layer L2, and the third layer L3 may be fixed (e.g., may be connected to or attached to each other), such that each of the pressure-sensitive layers 150 overlaps with one of crossing regions in which the first electrodes 110 cross the second electrodes 120 in a plan view. The first layer L1, the second layer L2, and the third layer L3 may be thermally fused with each other, or may be fixed by various suitable methods, such as an adhesive or sewing.

The pressure-sensitive layers 150 may include nanofibers coated with conductive particles. In an embodiment, the pressure-sensitive layers 150 may be formed by forming the partition wall 130 in a portion of the third nanofiber sheet 103, and reacting a conductive particle solution inside the holes 130h of the partition wall 130. The conductive particles may include carbon nanotubes, graphene, metal nanowires, or metal nanoflakes. The conductive particles may include (e.g., may be) carboxylic acid modified graphene, silver nanowires. or silver nanoflakes.

The third nanofiber sheet 103 may include integrated nanofibers formed using electrospinning, and may have a plurality of holes through which a gas or a fluid may pass. In an embodiment, the nanofibers may include polyurethane, polystyrene-block-poly(ethylene butylene)-block-polystyrene, polystyrene, polyvinyl chloride, or any suitable mixtures thereof. In an embodiment, the nanofibers may include (e.g., may be) polystyrene-block-poly(ethylene butylene)-block-polystyrene grafted with maleic anhydride (SEBS-g-MA).

As shown in FIG. 1, a j-th first electrode 110, an i-th second electrode 120, and the pressure-sensitive layer 150 disposed between the j-th first electrode 110 and the i-th second electrode 120 may form a pressure sensor cell Cij. The pressure sensor cells Cij may form an m×n matrix, and the pressure sensor array 10 may measure changes in electrical characteristics of each of the pressure sensor cells Cij to measure two-dimensional pressure changes based on the positions of the pressure sensor cells Cij. Here, m is the number of second electrodes 120, and is a natural number greater than 1. In addition, n is the number of first electrodes 110, and is a natural number greater than 1. Further, i is a natural number between 1 and m, and j is a natural number between 1 and n.

Respective pressure cells Cij are separated and fixed by the partition wall 130 to prevent or reduce electrical interferences between the pressure sensor cells Cij, even in a case where the pressure sensor array 10 is transformed.

Although FIGS. 1 to 5 show three first electrodes 110, three second electrodes 120, and nine pressure-sensitive layers 150, the present disclosure is not limited thereto, and the numbers thereof may be variously modified as needed or desired.

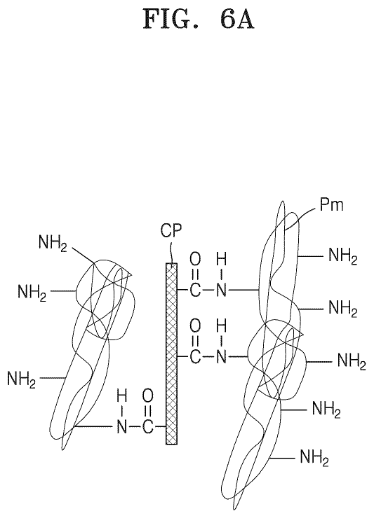

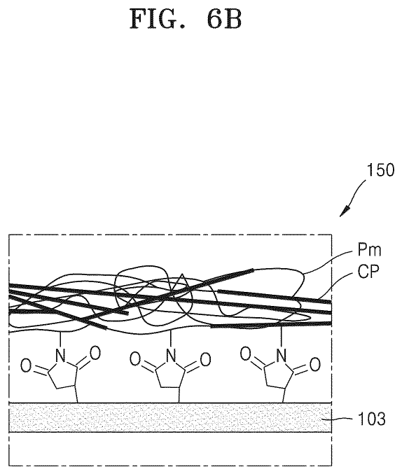

FIG. 6A is a view illustrating conductive particles. FIG. 6B is a view illustrating nanofibers coated with conductive particles.

Referring to FIG. 6A, conductive particles CP included in the pressure sensitive layer 150 (e.g., see FIG. 5) may be modified with (e.g., may be included in) a polymer Pm containing an amine functional group. As an example, the carboxyl group of the conductive particle CP and the amine functional group of the polymer (Pm) may react with each other (e.g., R-COOH+R′—NH2→R—CONH—R′+H2O), so that the conductive particle CP may be modified with (e.g., may be included in) the polymer Pm containing an amine functional group. The conductive particles CP may include carbon nanotubes, graphene, metal nanowires, or metal nanoflakes. The polymer Pm containing an amine functional group may include poly(allylamine), polyetherimide, poly(ethylene imine), and/or the like.

Referring to FIG. 6B, the conductive particles CP modified with (e.g., included in) the polymer Pm containing an amine functional group may be bonded to the nanofibers forming the third nanofiber sheet 103 through a chemical reaction. In an embodiment, the nanofibers may include polystyrene-block-poly(ethylene butylene)-block-polystyrene grafted with maleic anhydride (SEBS-g-MA). As shown in Reaction Scheme 1 below, the maleic anhydride group of the nanofiber (shown in Reaction Scheme 1 without the nanofiber) may undergo an imidization reaction with the amine functional group of the polymer Pm to form an imide bonded to both the nanofiber and the polymer Pm. In Reaction Scheme 1, the bond between the maleic anhydride and the nanofiber is not shown, and R of the amine functional group represents the polymer Pm.

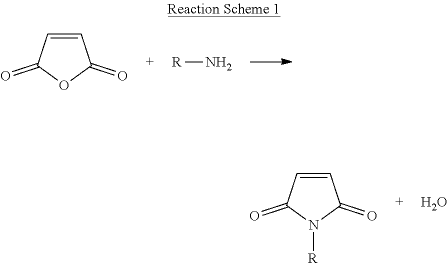

Accordingly, the nanofiber may be chemically bonded to the conductive particles through imide bonds. In a comparative example, when unmodified conductive particles are coated on nanofibers, the conductive particle coating may be easily cracked or peeled off when the nanofibers are transformed. On the other hand, according to some embodiments, because the pressure-sensitive layer 150 may be formed by coating nanofibers with conductive particles CP modified with (e.g., included in) the polymer Pm including an amine functional group, the nanofibers may be chemically bonded to the conductive particles. Accordingly, even in a case where the pressure-sensitive array 10 is repeatedly stretched or subjected to a strong pressure, the conductive particles CP may not be peeled off from the nanofibers, and may exhibit more stable device characteristics.

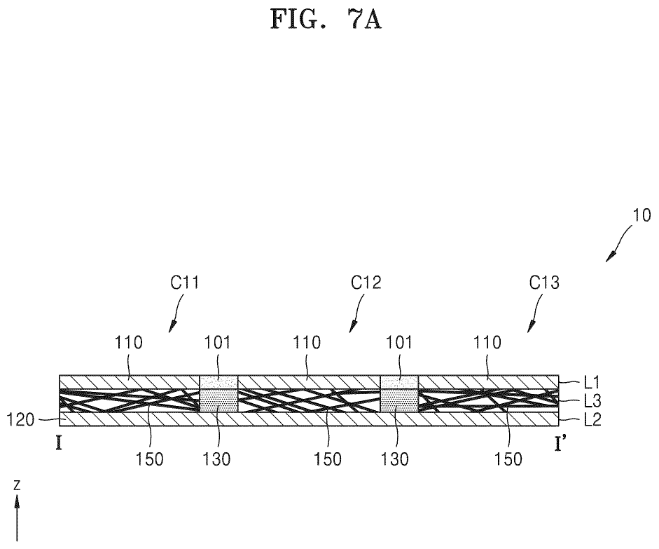

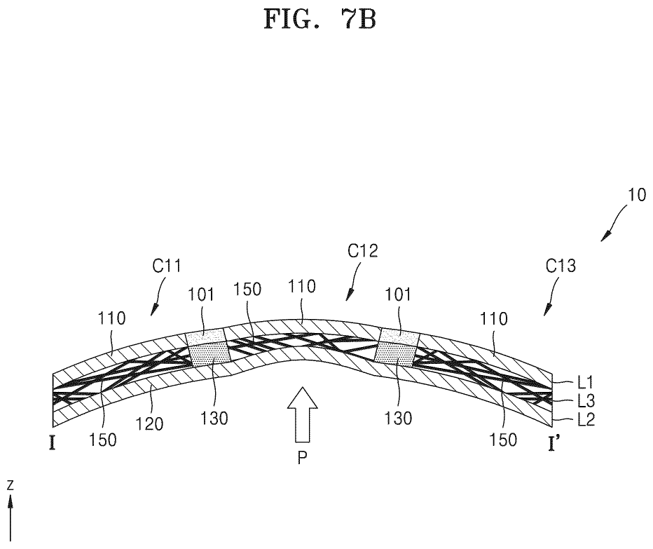

FIG. 7A is a schematic cross-sectional view of the pressure sensor array according to an embodiment. FIG. 7B is a view illustrating a case where a pressure is applied to the pressure sensor array of FIG. 7A.

FIG. 7A schematically shows a cross-section of the pressure sensor array 10 taken along the line I-I′ shown in FIG. 1.

Referring to FIG. 7A, the pressure sensor array 10 may include the first layer L1, the second layer L2, and the third layer L3. The first layer L1 may be disposed on the second layer L2, and the third layer L3 may be disposed between the first layer L1 and the second layer L2 (e.g., which includes the second electrode 120).

The first layer L1 may include the first electrodes 110 extending in the first direction (e.g., the y direction), and the second layer L2 may include the second electrodes 120 extending in the second direction (e.g., the x direction). The third layer L3 may include the partition wall 130 and the pressure-sensitive layers 150, which are disposed between the first electrode 110 and the second electrode 120.

The first layer L1, the second layer L2, and the third layer L3 may include (e.g., may be) nanofiber sheets including integrated nanofibers formed using electrospinning. The first electrodes 110 and the second electrodes 120 may be formed by coating nanofibers forming a nanofiber sheet with suitable metal materials. The pressure-sensitive layer 150 may be formed by forming the partition wall 130 with an insulating elastomer on the nanofiber sheet, and coating the nanofiber sheet inside the holes of the partition wall 130 with conductive particles.

Because each of the first layer L1, the second layer L2, and the third layer L3 has a network structure, each of the first layer L1, the second layer L2, and the third layer L3 may be transformed in various suitable directions. Accordingly, the pressure sensor array 10 may be stretched in one or more directions, and/or folded or rolled into a roll shape.

The pressure sensor cells Cij may be arranged to form a matrix. As an example, the pressure sensor cells Cij sharing one second electrode 120 may be arranged in the same row as each other, and the pressure sensor cells Cij sharing one first electrode 110 may be arranged in the same column as each other. For convenience of illustration, FIGS. 7A and 7B show a first pressure sensor cell C11, a second pressure sensor cell C12, and a third pressure sensor cell C13 that share one second electrode 120, and are arranged in the same row as each other.

One pressure sensor cell Cij may include the first electrode 110, the second electrode 120, and the pressure-sensitive layer 150 disposed between the first electrode 110 and the second electrode 120. The partition wall 130 separates the pressure-sensitive layers 150 from each other, and prevents or reduces electrical interferences between the pressure sensor cells Cij even when the pressure sensor array 10 is transformed into various shapes. In an embodiment, because the partition wall 130 may maintain or substantially maintain a relatively constant distance between the first electrode 110 and the second electrode 120 even when the pressure sensor array 10 is stretched, the partition wall 130 may reduce a change in current that may be caused by the stretching of the pressure sensor array 10.

The conductive particles of the pressure-sensitive layer 150 of the pressure sensor cell Cij may form a conductive path to electrically connect the first electrode 110 to the second electrode 120. When the pressure sensitive layer 150 is compressed, a number of conductive paths formed by the conductive particles may be increased, thereby reducing a resistance between the first electrode 110 and the second electrode 120. Accordingly, a pressure for each position of the pressure sensor array 10 may be calculated by measuring a current flowing in each of the pressure sensor cells Cij, or measuring a resistance between the first electrode 110 and the second electrode 120 forming the pressure sensor cell Cij.

Because each of the first electrode 110, the second electrode 120, and the pressure-sensitive layer 150 includes a plurality of holes, a gas or a fluid may easily pass through it. Accordingly, in a case where the first electrode 110, the second electrode 120, and the pressure-sensitive layer 150 are used as a respiratory sensing 1 sensor of a respiratory monitoring apparatus, a user may feel less foreign body sensations.

Referring to FIG. 7B, a pressure P may be applied to a portion of the pressure sensor array 10. As an example, a local pressure P may be applied to the second pressure sensor cell C12. The pressure sensor array 10 may be stretched in a thickness direction (e.g., the z direction), and may be entirely transformed by the pressure P.

The second pressure sensor cell C12 may be compressed by the pressure P, and conductive paths formed by the conductive particles included in the pressure-sensitive layer 150 of the second pressure sensor cell C12 may increase. Accordingly, a resistance between the first electrode 110 and the second electrode 120 forming the second pressure sensor cell C12 may be reduced, and a current flowing from the first electrode 110 to the second electrode 120 may be increased. As such, the pressure P applied to the second pressure sensor cell C12 may be measured based on a change in a resistance value or a change in a current value.

The first pressure sensor cell C11 and the third pressure sensor cell C13 may be at least partially stretched by the transformation of the pressure sensor array 10. In a case where the pressure P is not directly applied to the first pressure sensor cell C11 and the third pressure sensor cell C13, the thicknesses of the first pressure sensor cell C11 and the third pressure sensor cell C13 may be maintained or substantially maintained by the partition wall 130. Accordingly, a resistance of the pressure-sensitive layer 150 of each of the first pressure sensor cell C11 and the third pressure sensor cell C13 may not change or may change relatively little (e.g., may be relatively less than that where the pressure P is directly applied). As such, the pressure sensor array 10 may more accurately measure a position at which a local pressure P is applied, and the intensity of the pressure P.

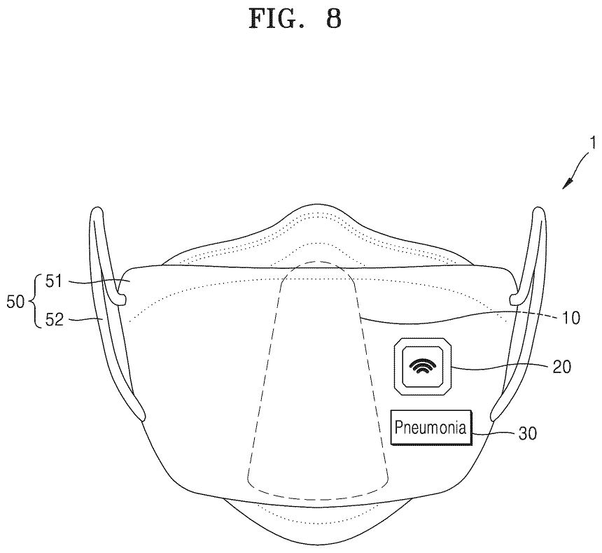

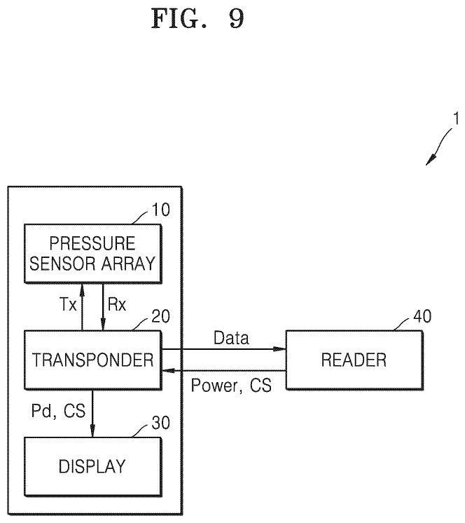

FIG. 8 is a schematic view of a portion of a respiratory monitoring apparatus 1 according to an embodiment. FIG. 9 is a schematic block view of the respiratory monitoring apparatus 1 according to an embodiment.

Referring to FIGS. 8 and 9, the respiratory monitoring apparatus 1 according to an embodiment may include the pressure sensor array 10, a transponder 20, a display 30, a reader 40, and a mask portion 50.

The mask portion 50 may be a means to fix the pressure sensor array 10, the transponder 20, and the display 30 to a user's face. The mask portion 50 may include a body 51 that surrounds (e.g., around) the user's face, and a fixing portion 52 for fixing the body 51 to the user's ears or head. In an embodiment, the body 51 of the mask portion 50 may include a flexible, breathable sheet, and may be in close contact with the user's face.

The pressure sensor array 10 may be fixed to cover the user's nose and mouth by the mask portion 50. The pressure sensor array 10 may be disposed on the central portion of the body 51 of the mask portion 50, and may be disposed on the rear surface of the body 51, or in other words, towards the user's face. As described above with reference to FIGS. 1 to 5, the pressure sensor array 10 may include the plurality of pressure sensor cells Cij to measure changes in pressure for each position caused by the user's respiration.

In an embodiment, the pressure sensor array 10 may be integrated with the mask portion 50. As an example, the pressure sensor array 10 may be integrated with the body 51 of the mask portion 50. In another embodiment, the pressure sensor array 10 may be detachably provided to the mask portion 50. As described above, the pressure sensor array 10 may include a nanofiber sheet including the plurality of holes, and may be transformable in various suitable shapes. Accordingly, the pressure sensor array 10 may be in close contact with the user's face, and may reduce a feeling of discomfort during breathing.

The transponder 20 may supply voltages to the pressure sensor array 10 and the display 30, may apply a driving signal Tx to the pressure sensor array 10, and may convert a sensing signal Rx received from the pressure sensor array 10 into a data signal Data to transmit the data signal Data to the reader 40. In an embodiment, a sensing signal Rx may include (e.g., may be) a change in a resistance value or a change in a current value of each pressure sensor cell included in the pressure sensor array 10. The transponder 20 may be provided in a chip form, and may be fixed to the user's face by the mask portion 50.

The reader 40 may supply power to the transponder 20 through an inductive coupling, and may receive a data signal Data from the transponder 20 to analyze the data signal Data. The reader 40 may be a computing device that analyzes a data signal Data. As an example, the reader 40 may include a memory for storing the data signals Data, commands, software, applications, and the like, and a processor to process the data signals Data and perform various suitable operations.

In an embodiment, the reader 40 may analyze the user's respiration pattern based on the data signals Data, and may generate control signals CS including the user's respiration information. A data signal Data may include a two-dimensional pressure change due to the user's respiration that is measured using the pressure sensor array 10. Depending on the location of the pressure change, oral breathing and nasal breathing may be distinguished. The reader 40 may obtain the user's breathing pattern data over time through the location of the pressure change and the intensity of the pressure. In an embodiment, the reader 40 may generate respiration information including information corresponding to a respiratory disease of the user or the user's breathing habits from the breathing pattern data. The reader 40 may transfer a control signal CS to the transponder 20.

The transponder 20 may transfer, to the display 30, a voltage Pd and a control signal CS for driving the display 30. The display 30 may display the user's respiration information based on the control signal CS. In an embodiment, the display 30 may include a reflective display panel that does not use a separate battery, and a display circuit to control the display panel.

In an embodiment the display 30 may include a self-luminous display panel. The self-luminous display panel may include an organic light-emitting diode OLED, an inorganic light-emitting diode, a quantum-dot light-emitting diode QED, a micro light-emitting diode, a nano light-emitting diode, and/or the like. Because the self-luminous display panel may not include a separate transistor, the self-luminous display panel may be provided as a passive matrix display panel with a relatively low power consumption. In an embodiment, the display 30 may include a flexible display panel that may be transformed.

The display 30 may be fixed to the user's face by the mask portion 50. The user may simply check the respiratory information, such as the respiratory disease or the breathing habits, through the display 30. In an embodiment, the display 30 may be omitted as needed or desired.

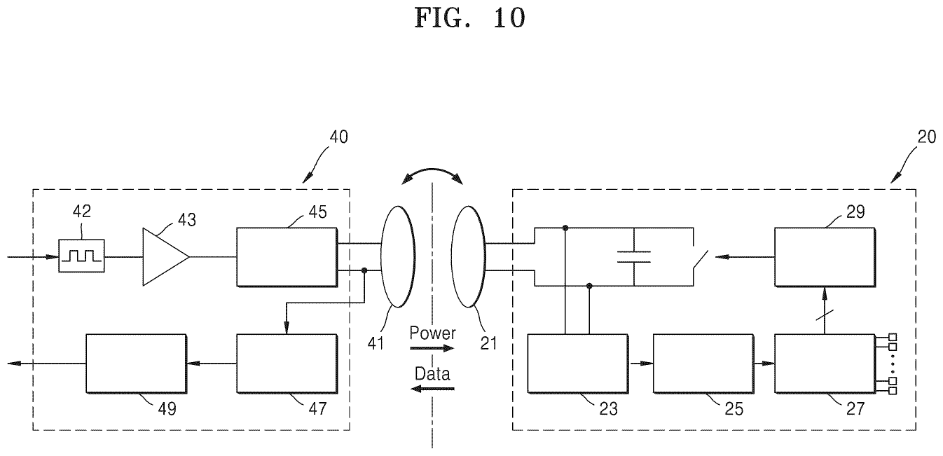

FIG. 10 is a view of the transponder 20 and the reader 40.

Referring to FIG. 10, the transponder 20 and the reader 40 may form a wireless communication system configured to transfer voltages and data signals using load shift keying.

The transponder 20 may include a first antenna 21, a rectifier 23, a power manager 25, a pressure sensor reader 27, and a data serializer 29. The transponder 20 may receive power from the reader 40 through an inductive coupling of the first antenna 21 and a second antenna 41 of the reader 40.

The rectifier 23 may be connected to both ends (e.g., opposite ends) of the first antenna 21, and may convert an alternating current voltage received through the first antenna 21 into a direct current voltage. The power manager 25 may receive a direct current voltage from the rectifier 23, and may supply a voltage to the pressure sensor array 10, the display 30, and other elements of the transponder 20. The power 1 manager 25 may be provided in the form of a power management integrated circuit that is embedded in the transponder 20.

The pressure sensor reader 27 may include a control circuit to control the pressure sensor array 10 (e.g., see FIG. 9), and an analog-digital converter to convert an analog signal received from the pressure sensor array 10 into a first signal, which may be a digital signal. The pressure sensor reader 27 may receive a voltage from the power manager 25, may apply a driving signal Tx (e.g., see FIG. 9) to the pressure sensor array 10, may detect a sensing signal Rx, and may convert the sensing signal Rx into a first signal, which is the digital signal.

The data serializer 29 may serialize a first signal transferred from the pressure sensor reader 27, and may convert the first signal into a data signal Data. The data signal Data may include address information indicating the position of each measurement value. The data serializer 29 may turn on/off a switch according to a data signal to change a voltage level of the first antenna 21.

The reader 40 may include the second antenna 41, an amplifier 43, a matching network 45, a signal processor 47, and a data demodulator 49.

The amplifier 43 may generate an alternating current voltage having a desired frequency (e.g., a preset or predetermined frequency) according to a clock signal 42 input from the outside. Power generated by the amplifier 43 may be transferred to the second antenna 41 through the matching network 45. The matching network 45 may reduce a loss caused by a voltage transmission through impedance matching.

The signal processor 47 may be connected to the second antenna 41, and may sense a voltage level of the first antenna 21. As described above, the data serializer 29 of the transponder 20 may turn on/off the switch according to the data signal Data to change the voltage level of the first antenna 21, and a change in the voltage level of the first antenna 21 may change an impedance of a circuit connected to the second antenna 41 through inductive coupling. The signal processor 47 may 1 sense a voltage level change of the first antenna 21 from a voltage swing width difference due to an impedance change, and may transfer the voltage level change to the data demodulator 49.

The data demodulator 49 may demodulate a data signal from the voltage level change of the first antenna 21 received from the signal processor 47 using amplitude shift keying.

Because the reader 40 may generate an alternating current voltage of a sinusoidal wave form for wireless communication and transfer the alternating current voltage to the first antenna 21 of the transponder 20, the transponder 20 may operate without using a separate battery. In this case, a frequency of the alternating current power source used may be about 433 MHZ. Accordingly, because the size of the first antenna 21 may be reduced, the user's discomfort that may be caused by the transponder 20 may be reduced. In addition, even when a data signal Data is transferred from the transponder 20 to the reader 40, the reader 40 may concurrently or substantially simultaneously supply an induced voltage to the transponder 20.

Hereinafter, an experimental example according to some embodiments will be described in more detail.

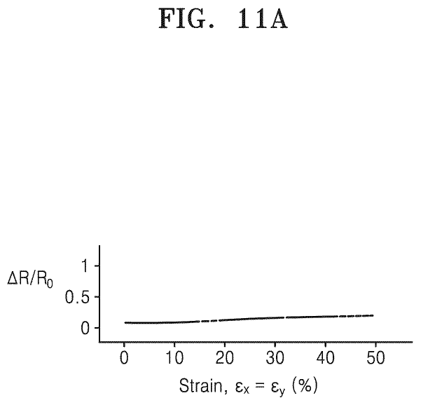

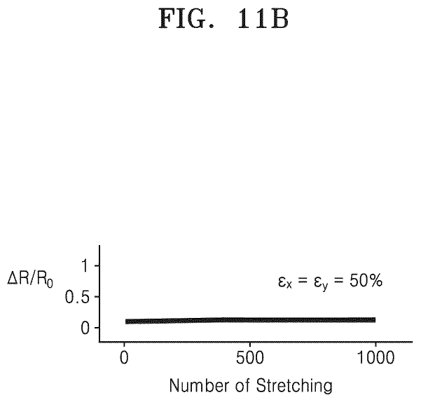

FIG. 11A is a graph showing a relationship between an elongation and a rate of change in resistance of a nanofiber electrode coated with a metal. FIG. 11B is a graph showing a relationship between the number of stretching repetitions and a rate of change in resistance of a nanofiber electrode coated with a metal.

The metal-coated nanofiber electrode may be formed by sputtering gold (Au) onto a styrene-ethylene-butylene-styrene nanofiber sheet grafted with maleic anhydride, which may be formed using electrospinning.

Referring to FIG. 11A, the nanofiber sheet is stretched in the first direction (e.g., the y direction) and the second direction (e.g., the x direction), and the resistance of the metal-coated nanofiber electrode is measured. In this case, even when the 1 elongation of the nanofiber sheet is increased up to 50%, the resistance of the metal-coated nanofiber electrode may not be changed significantly.

Referring to FIG. 11B, the resistance of the metal-coated nanofiber electrode may be measured while stretching the nanofiber sheet by about 50% in the first direction (e.g., the y direction) and the second direction (e.g., the x direction), and then restoring the nanofiber sheet to its original state. Although the number of stretching repetitions may be increased to 1,000 times, the resistance of the metal-coated nanofiber electrode may not be changed significantly.

Because the first electrode and the second electrode of the pressure sensor array may include the metal-coated nanofiber electrode, the resistance of the first electrode and the second electrode may not be changed significantly. Accordingly, the respiratory monitoring apparatus according to some embodiments may distinguish and sense a user's breathing from the transformation of the pressure sensor array, for example, such as due to the user's movement and the like.

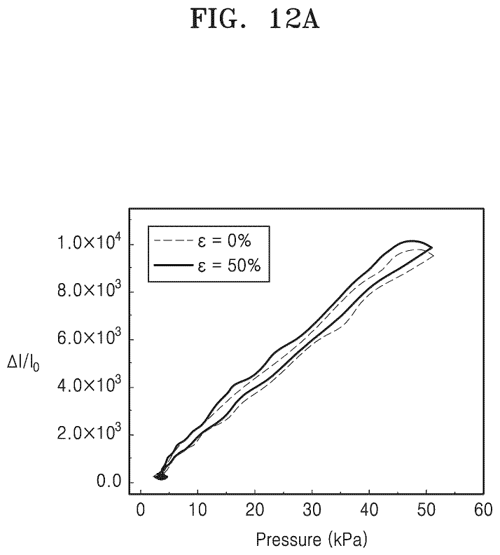

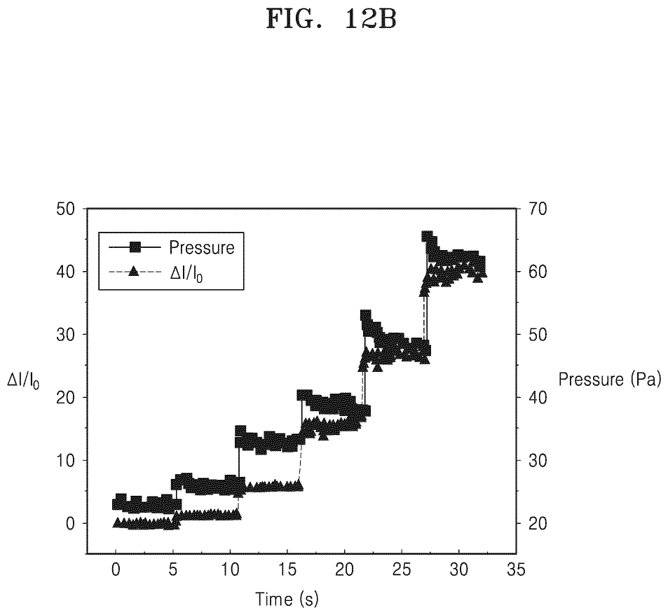

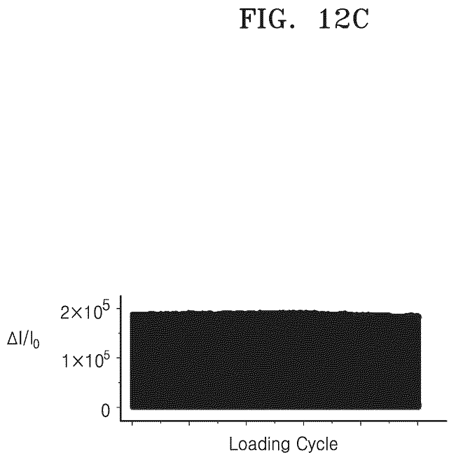

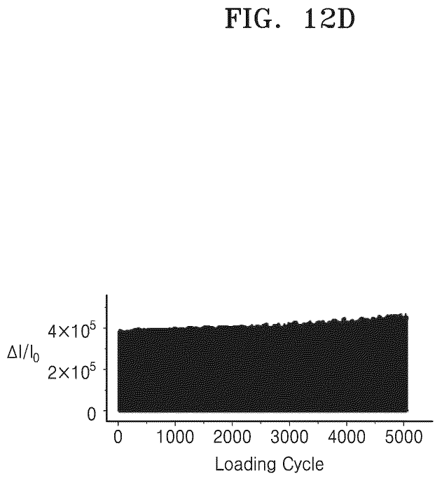

FIG. 12A is a graph showing a relationship between a pressure and a rate of change in current in each of an unstretched pressure sensor and a stretched pressure sensor. FIG. 12B is a graph showing a pressure and a rate of change in current of a pressure sensor measured in real time. FIG. 12C is a graph showing a relationship between a loading cycle and a rate of change in current of an unstretched pressure sensor. FIG. 12D is a graph showing a relationship between a loading cycle and a rate of change in current of a stretched pressure sensor.

The first layer including the first electrodes and the second layer including the second electrodes may be formed by sputtering gold (Au) onto styrene-ethylene-butylene-styrene nanofiber sheets grafted with maleic anhydride formed using electrospinning. A partition wall including a plurality of holes may be formed on the styrene-ethylene-butylene-styrene nanofiber sheet grafted with maleic anhydride using an elastomer, and a pressure sensor array may be manufactured by reacting a carbon nanotube solution modified with polyethyleneimine inside a plurality of holes to form a 1 pressure-sensitive layer. FIGS. 12A to 12D show results of measuring a current flowing through a pressure sensor cell forming one cell of the pressure sensor array.

Referring to FIG. 12A, when a pressure applied to an unstretched pressure sensor and a 50% stretched pressure sensor is increased to 50 kPa and then decreased, a current change rate may be increased and then decreased in proportion to the pressure. In this case, the current change rates of the unstretched pressure sensor and the 50% stretched pressure sensor may have the same or substantially the same pressure sensitive characteristic as each other.

Referring to FIG. 12B, when pressure applied to the pressure sensor is increased at suitable time intervals (e.g., at preset or predetermined time intervals), the current change rate of the pressure sensor may be increased correspondingly. The respiratory monitoring apparatus according to an embodiment may calculate a change in the pressure due to a user's breathing from the current change rate of the pressure sensor.

FIG. 12C shows a current change rate of the unstretched pressure sensor when repeating a loading cycle of increasing and decreasing the pressure, and FIG. 12D shows a current change rate of the pressure sensor stretched by about 50% when repeating the loading cycle. Referring to FIGS. 12C and 12D, both the unstretched pressure sensor and the 50% stretched pressure sensor may exhibit stable pressure sensitive characteristics while the loading cycle is repeated 5,000 times. Although the pressure sensor array and the respiratory monitoring apparatus have been mainly described, the present disclosure is not limited thereto. As an example, a respiratory monitoring method using the pressure sensor array and the respiratory apparatus may also fall within the spirit and scope of the present disclosure.

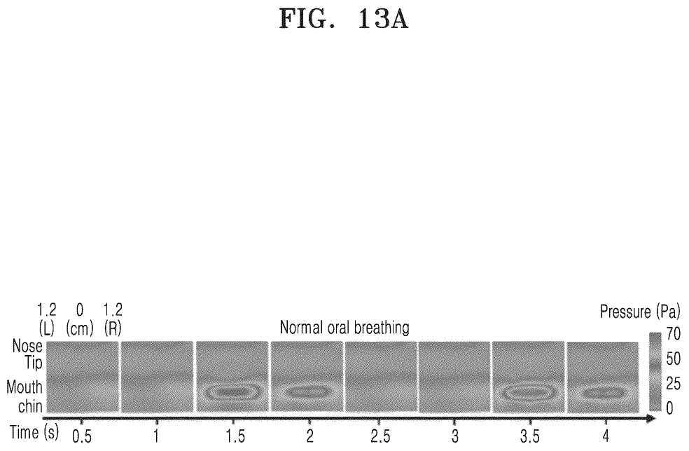

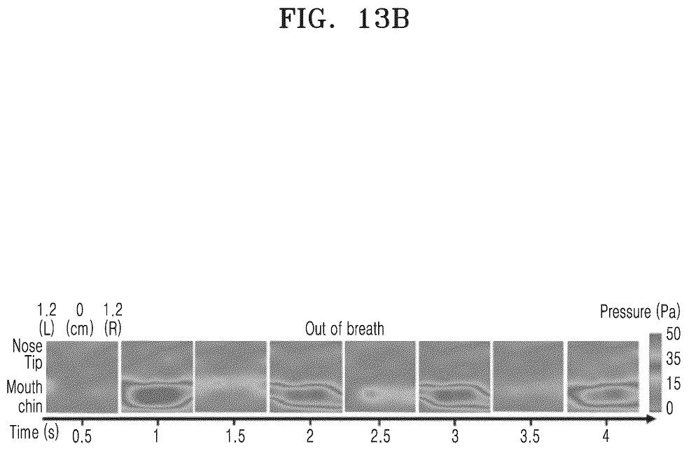

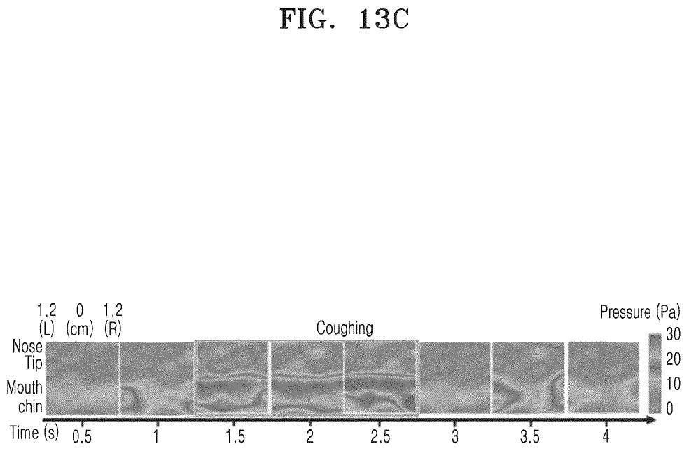

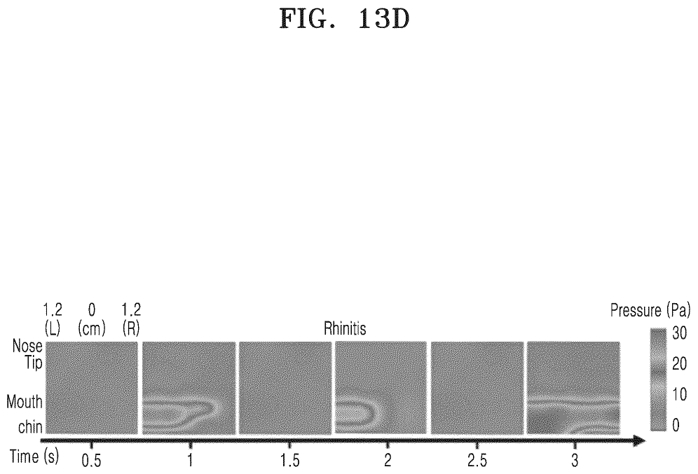

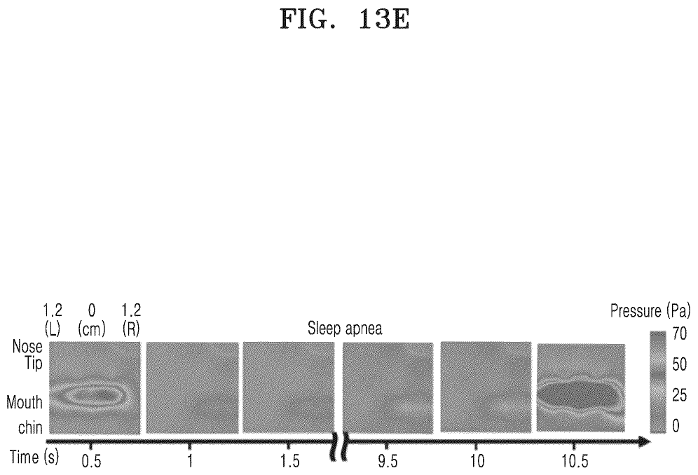

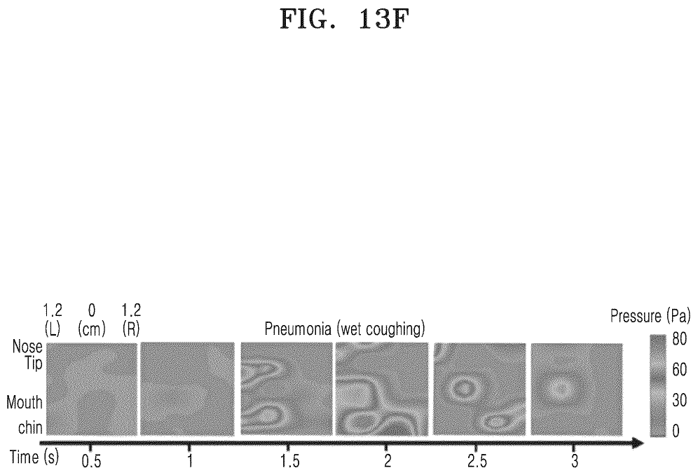

FIGS. 13A through 13F are graphs showing a pressure change in the pressure sensor array measured using the respiratory monitoring apparatus according to one or more embodiments.

Referring to FIGS. 13A to 13F, the respiratory monitoring apparatus may two-dimensionally measure the pressure change in each cell of the pressure sensor array over time. As an example, a pressure change measured on the upper side of the pressure sensor array may be due to the user's nasal breathing, and a pressure change measured on the lower side of the pressure sensor array may be due to the user's oral breathing.

FIG. 13A shows a pressure change of the pressure sensor array due to normal oral breathing measured over time. FIG. 13B shows a pressure change of the pressure sensor array measured over time when the user is out of breath (e.g., when the user is having difficulty breathing). FIG. 13C shows a pressure change of the pressure sensor array measured over time when the user coughs dryly. FIG. 13D shows a pressure change of the pressure sensor array due to a respiration of a rhinitis patient measured over time. FIG. 13E shows a pressure change of the pressure sensor array due to breathing during sleep of a sleep apnea patient measured over time. FIG. 13F shows a pressure change of the pressure sensor array due to a respiration of a pneumonia patient with a wet cough measured over time. Depending on the user's condition, a pressure change location, a pressure intensity, and/or a pressure change frequency may appear different.

The respiratory monitoring apparatus may measure a pressure change due to the user's breathing two-dimensionally over time to obtain the user's breathing pattern, and from this, may analyze the user's breathing habits and respiratory diseases. As described above, the breathing monitoring apparatus according to some embodiments may be fixed to the user's face by the mask portion and may operate without using a separate battery, enabling long-term breathing pattern analysis without interfering with the user's activities. In addition, because each layer of the pressure sensor array may include a breathable nanofiber sheet with a plurality of holes, foreign body sensations may be reduced when the user breathes.

According to some embodiments described above, a stretchable pressure sensor array that may be transformable into various suitable shapes, and the respiratory monitoring apparatus including the pressure sensor array may be implemented. However, the present disclosure is not limited thereto.

The electronic or electric devices and/or any other relevant devices or components according to embodiments of the present disclosure described herein may be implemented utilizing any suitable hardware, firmware (e.g. an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

The foregoing is illustrative of some embodiments of the present disclosure, and is not to be construed as limiting thereof. Although some embodiments have been described, those skilled in the art will readily appreciate that various modifications are possible in the embodiments without departing from the spirit and scope of the present disclosure. It will be understood that descriptions of features or aspects within each embodiment should typically be considered as available for other similar features or aspects in other embodiments, unless otherwise described. Thus, as would be apparent to one of ordinary skill in the art, features, characteristics, and/or elements described in connection with a particular embodiment may be used singly or in combination with features, characteristics, and/or elements described in connection with other embodiments unless otherwise specifically indicated. Therefore, it is to be understood that the foregoing is illustrative of various example embodiments and is not to be construed as limited to the specific embodiments disclosed herein, and that various modifications to the disclosed embodiments, as well as other example embodiments, are intended to be included within the spirit and scope of the present disclosure as defined in the appended claims, and their equivalents.

What is claimed is:

1. A pressure sensor array comprising:
a first sheet comprising first electrodes extending in a first direction and spaced from each other along a second direction crossing the first direction;
a second sheet comprising second electrodes extending in the second direction and spaced from each other along the first direction; and
a third sheet between the first sheet and the second sheet, and comprising:
a partition wall defining a plurality of holes that are spaced from each other in the first direction and the second direction; and
pressure-sensitive layers located on an inner surface of each of the plurality of holes.

2. The pressure sensor array of claim 1, wherein each of the first sheet, the second sheet, and the third sheet comprises nanofibers.

3. The pressure sensor array of claim 1, wherein each of the first electrodes and the second electrodes comprises nanofibers coated with a metal.

4. The pressure sensor array of claim 1, wherein the partition wall comprises an elastomer.

5. The pressure sensor array of claim 1, wherein each of the pressure-sensitive layers comprises nanofibers coated with conductive particles.

6. The pressure sensor array of claim 5, wherein the nanofibers are bonded to the conductive particles through imide bonding.

7. The pressure sensor array of claim 1, wherein each of the pressure-sensitive layers overlaps with a corresponding one of a plurality of crossing regions in which the first electrodes cross the second electrodes.

8. A respiratory monitoring apparatus comprising:
a pressure sensor array configured to sense pressure changes caused by a user's breathing;
a transponder configured to supply a voltage to the pressure sensor array, convert a sensing signal received from the pressure sensor array into a data signal, and transmit the converted data signal;
a reader configured to supply a voltage to the transponder through inductive coupling, and receive the data signal from the transponder; and
a mask portion configured to fix the pressure sensor array and the transponder to the user's face,
wherein the pressure sensor array comprises:
a first sheet comprising first electrodes extending in a first direction and spaced from each other along a second direction crossing the first direction;
a second sheet comprising second electrodes extending in the second direction and spaced from each other along the first direction; and
a third sheet between the first sheet and the second sheet, and comprising:
a partition wall defining a plurality of holes that are spaced from each other in the first direction and the second direction; and
pressure-sensitive layers located on an inner surface of each of the plurality of holes.

9. The respiratory monitoring apparatus of claim 8, wherein each of the first sheet, the second sheet, and the third sheet comprises nanofibers.

10. The respiratory monitoring apparatus of claim 8, wherein each of the first electrodes and the second electrodes comprises nanofibers coated with a metal.

11. The respiratory monitoring apparatus of claim 8, wherein the partition wall comprises an elastomer.

12. The respiratory monitoring apparatus of claim 8, wherein each of the pressure-sensitive layers comprises nanofibers coated with conductive particles.

13. The respiratory monitoring apparatus of claim 12, wherein the nanofibers are bonded to the conductive particles through imide bonding.

14. The respiratory monitoring apparatus of claim 8, wherein each of the pressure-sensitive layers overlaps with a corresponding one of a plurality of crossing regions in which the first electrodes cross the second electrodes.

15. The respiratory monitoring apparatus of claim 8, wherein the transponder comprises:
a first antenna;
a rectifier configured to convert an alternating current voltage received through the first antenna into a direct current voltage;
a pressure sensor reader configured to input a driving signal to the pressure sensor array, receive a sensing signal from the pressure sensor array, and convert the sensing signal into a first signal;
a data serializer configured to serialize the first signal, convert the serialized first signal into the data signal, and change a voltage level of the first antenna according to the data signal; and
a power manager configured to receive the direct current voltage from the rectifier, and distribute the direct current voltage.

16. The respiratory monitoring apparatus of claim 15, wherein the reader comprises:
a second antenna;
an amplifier configured to generate the alternating current voltage according to an input clock signal;
a matching network configured to transfer the alternating current voltage from the amplifier to the second antenna;
a signal processor configured to sense a voltage level of the first antenna; and
a data demodulator configured to demodulate the data signal from the voltage level of the first antenna.

17. The respiratory monitoring apparatus of claim 16, wherein the alternating current voltage comprises a sinusoidal wave of about 433 MHZ.

18. The respiratory monitoring apparatus of claim 8, further comprising a display connected to the mask portion, and configured to display a user's respiratory information.

19. The respiratory monitoring apparatus of claim 18, wherein the display comprises a reflective display panel.

20. The respiratory monitoring apparatus of claim 18, wherein the reader is configured to generate, based on the data signal, a control signal comprising the user's respiratory information, and transfer the control signal to the transponder, and
wherein the transponder is configured to transfer the control signal to the display.
